(19) United States
(12) Patent Application Publication
(10) Pub. No.: US 2016/0053317 A1
(43) Pub. Date: Feb. 25, 2016

(54) FETAL DIAGNOSTICS USING FETAL CELL CAPTURE FROM MATERNAL BLOOD

(71) Applicant: Basetra Medical Technology Co. Ltd., Shanghai (CN)

(72) Inventors: Han Wu, Albany, CA (US); Fanqing Chen, Moraga, CA (US)

(21) Appl. No.: 14/784,943

(22) PCT Filed: May 15, 2014
(86) PCT No.: PCT/US2014/038250

§ 371 Date: Oct. 15, 2015

Related U.S. Application Data
(60) Provisional application No. 61/824,128, filed on May 16, 2013.

(51) Int. Cl. C12Q 1/68; G01N 33/569; G01N 33/573

(52) U.S. Cl. CPC C12Q 1/6881; G01N 33/573; G01N 33/56966; G01N 2333/96494

(57) ABSTRACT

Non-invasive fetal diagnostic methods are provided. In particular, provided are methods of obtaining a fetal cell-enriched sample from a maternal sample and methods of assessing a maternal sample for a fetal nucleotide sequence or expression of a fetal gene.

### INCORPORATION BY REFERENCE TO ANY PRIORITY APPLICATIONS

The present application claims priority under 35 U.S.C. §119(e) to U.S. Provisional Application No. 61/824,128, filed May 16, 2013, which is herein expressly incorporated by reference in its entirety.

### BACKGROUND

Prenatal diagnostics can provide helpful information regarding the state of a fetus. Typically, prenatal diagnostics are performed using invasive techniques that posed a risk for the fetus.

More recently, fetal genetic material has been found within the mother's circulating blood. This fetal genetic material originates in the fetus and crosses the placenta to enter the mother's circulatory system.

The two major noninvasive resources of fetal DNA are the maternal plasma and the circulating fetal cells. Fetal genetic material can be detected in maternal blood early in gestation. Several non-invasive diagnosis strategies have been developed by taking advantage of cell-free fetal DNA. According to quantitate assays, however, the fetal DNA only accounts for ˜3.4% and ˜6.2% of the total plasma. DNA in early and late pregnancy, respectively. This is technically challenging for accurate assays since half of the genetic information of fetus originates from the father, which will be overwhelmed by the maternal DNA. Additionally, since fetal cell-free DNA is not protected by the cell membrane, the DNA fragments are short and incomplete compared with the intact genome. As for the fetal cells from maternal blood, it is also difficult for isolation due to the scarcity of their existence, which can be as low as the order of 1 fetal cell in 10⁶˜10⁷ maternal cells. In order to obtain fetal cells sufficient for analysis, the use of enrichment techniques are. Due to their low numbers, it is technically challenging to enrich and purify a fetal cell from maternal blood samples. These challenges ultimately complicate fetal diagnostic methods.

### SUMMARY OF THE INVENTION

The inventors have developed improved non-invasive fetal diagnostic methods. In particular, provided are methods of obtaining a fetal cell-enriched sample from a maternal sample and methods of assessing a maternal sample for a fetal nucleotide sequence or expression of a fetal gene.

In some embodiments, provided herein is a method of obtaining a fetal cell-enriched sample from a maternal sample comprising: providing a maternal sample; contacting the maternal sample with a first stationary phase having affinity for one or more saccharides; separating components of the maternal sample bound to the first stationary phase from components of the maternal sample not bound to the first stationary phase; retaining the components of the maternal sample bound to the first stationary phase; contacting the maternal sample with an isolatably labeled affinity molecule having affinity for matrix metalloproteinase 14; separating components of the maternal sample bound to the isolatably labeled affinity molecule from components of the maternal sample not bound to the isolatably labeled affinity molecule; and retaining the components of the maternal sample bound to the isolatably labeled affinity molecule, thereby providing a fetal cell-enriched sample. Some such embodiments further comprise, prior to contacting the maternal sample with said first stationary phase and said first stationary phase: separating components of the maternal sample according to size and/or density; and harvesting the separated components of the maternal sample having the size and/or density of nucleated fetal red blood cells. In some embodiments, the separating components of the maternal sample according to size and/or density is performed using gradient centrifugation. In some embodiments, the one or more saccharides is galactose. In some embodiments, the first stationary phase is a lectin-bound stationary phase. In some embodiments, the first stationary phase comprises a magnetic bead. In some embodiments, the isolatably labeled affinity molecule is bound to a magnetic bead. In some embodiments, subsequent to retaining the components of the maternal sample bound to the first stationary phase, the retained components of the maternal sample bound to the first stationary phase are contacted with the isolatably labeled affinity molecule having affinity for matrix metalloproteinase 14. Some embodiments further comprise contacting the maternal sample with a second stationary phase having affinity for a fetal cell surface marker other than matrix metalloproteinase 14; separating components of the maternal sample bound to the second stationary phase from components of the maternal sample not bound to the second stationary phase; and retaining the components of the maternal sample bound to the second stationary phase. In some embodiments, the fetal cell surface marker is selected from the group consisting of transferin receptor (CD71), glycophorin A (GPA), HLA-G, EGFR, thrombospondin receptor (CD36), CD 34, HbF, HAE 9, FB3-2, H3-3, erythropoietin receptor, HBE, AFP, APOC3, SERPINC1, AMBP, CPB2, ITIH1, APOH, HPX, beta-hCG, AHSG, APOB, J42-4-d, 2,3-biophosphoglycerate (BPG), Carbonic anhydrase (CA), and Thymidine kinase (TK). In some embodiments, the fetal cell surface marker is transferin receptor (CD71). In some embodiments, the maternal sample is a maternal blood sample. In some embodiments, at least 50%, 60%, 70%, 80%, or 90% of the cells in the fetal cell-enriched sample are fetal cells. Some embodiments further comprise providing the fetal cell-enriched sample; and analyzing a nucleotide sequence of a nucleic acid molecule or expression of a gene in one or more cells from the fetal cell-enriched sample. In some embodiments, the analyzing a nucleotide sequence of a nucleic acid molecule comprises sequencing genomic DNA of one or more cells from the fetal cell-enriched sample. In some embodiments, the sequencing genomic DNA comprises sequencing the DNA of a single cell, and wherein sequencing the DNA of a single cell is performed for one or more cells from the fetal cell-enriched sample. In some embodiments, sequencing the DNA of a single cell is performed for at least 2, 3, 4, 5, 6, 7, 8, 9 or 10 cells from the fetal cell-enriched sample. In some embodiments, the expression of a gene comprises hybridizing a detectable antibody to the surface of one or more cells from the fetal cell-enriched sample. In some embodiments, the analyzing a nucleotide sequence of a nucleic acid molecule comprises hybridizing a detectable probe to the genomic DNA of one or more cells from the fetal cell-enriched sample. In some embodiments, one or more individual cells are analyzed for hybridization of a detectable probe to the genomic DNA of each analyzed cell. In some embodiments, at least 2, 3, 4, 5, 6, 7, 8, 9 or 10 individual cells are analyzed for hybridization of a detectable probe to the genomic DNA of each analyzed cell.

Also provided herein are methods of assessing a maternal sample for a fetal nucleotide sequence or a fetal gene expression comprising: providing a maternal sample; contacting the maternal sample with a first stationary phase having affinity for one or more saccharides; separating components of the maternal sample bound to the first stationary phase from components of the maternal sample not bound to the first stationary phase; retaining the components of the maternal sample bound to the first stationary phase; contacting the maternal sample with an isolatably labeled affinity molecule having affinity for a fetal cell surface marker; separating components of the maternal sample bound to the isolatably labeled affinity molecule from components of the maternal sample not bound to the isolatably labeled affinity molecule; retaining the components of the maternal sample bound to the isolatably labeled affinity molecule to provide an fetal cell-enriched sample for analysis, determining a nucleotide sequence of a nucleic acid molecule or expression of a gene in individual cells for each of two or more cells of the fetal cell-enriched sample; and assessing the nucleotide sequence of a nucleic acid molecule or expression of a gene determined for individual cells for each of two or more cells of the fetal cell-enriched sample to identify a fetal nucleotide sequence or gene expression. In some embodiments, assessing the nucleotide sequence of a nucleic acid molecule or expression of a gene determined for individual cells for each of two or more cells of the fetal cell-enriched sample comprises: classifying each cell as belonging to a first population of cells or a second population of cells based on the determined nucleotide sequence or gene expression; and identifying probabilities of the first and second populations of cells being of fetal or maternal origin. In some embodiments, the probabilities of the first and second populations of cells being of fetal or maternal origin are identified by comparing the nucleotide sequence of a nucleic acid molecule or expression of a gene for each of the first and second populations to a known nucleotide sequence of a nucleic acid molecule or expression of a gene for known maternal cells, wherein the population of cells bearing a higher nucleotide sequence or gene expression similarity to the known maternal cells is identified as being of maternal origin. In some embodiments, the probabilities of the first and second populations of cells being of fetal or maternal origin are identified by assessing the size of the first and second populations of cells, wherein the larger population of cells is identified as being of fetal origin.

Also provided are methods of assessing a maternal sample for a fetal nucleotide sequence or a fetal gene expression comprising: providing a nucleotide sequence of a nucleic acid molecule or expression of a gene in individual cells for each of two or more cells of the fetal cell-enriched sample, wherein the fetal cell-enriched sample has been prepared by a method comprising: providing a maternal sample; contacting the maternal sample with a first stationary phase having affinity for one or more saccharides; separating components of the maternal sample bound to the first stationary phase from components of the maternal sample not bound to the first stationary phase; retaining the components of the maternal sample bound to the first stationary phase; contacting the maternal sample with an isolatably labeled affinity molecule having affinity for a fetal cell surface marker; separating components of the maternal sample bound to the isolatably labeled affinity molecule from components of the maternal sample not bound to the isolatably labeled affinity molecule; and retaining the components of the maternal sample bound to the isolatably labeled affinity molecule to provide an fetal cell-enriched sample for analysis; and assessing the nucleotide sequence of a nucleic acid molecule or expression of a gene determined for individual cells for each of two or more cells of the fetal cell-enriched sample to identify a fetal nucleotide sequence or gene expression. In some embodiments, the probabilities of the first and second populations of cells being of fetal or maternal origin are identified by comparing the nucleotide sequence of a nucleic acid molecule or expression of a gene for each of the first and second populations to a known nucleotide sequence of a nucleic acid molecule or expression of a gene for known maternal cells, wherein the population of cells bearing a higher nucleotide sequence or gene expression similarity to the known maternal cells is identified as being of maternal origin. In some embodiments, the probabilities of the first and second populations of cells being of fetal or maternal origin are identified by assessing the size of the first and second populations of cells, wherein the larger population of cells is identified as being of fetal origin. In some embodiments, the fetal cell-enriched sample preparation method further comprises, prior to contacting the maternal sample with said first stationary phase and said first stationary phase: separating components of the maternal sample according to size and/or density; and harvesting the separated components of the maternal sample having the size and/or density of nucleated fetal red blood cells.

In some embodiments of the methods provided herein, the separating components of the maternal sample according to size and/or density is performed using gradient centrifugation. In some embodiments, the one or more saccharides is galactose. In some embodiments, the first stationary phase is a lectin-bound stationary phase. In some embodiments, the first stationary phase comprises a magnetic bead. In some embodiments, the isolatably labeled affinity molecule is bound to a magnetic bead. In some embodiments, subsequent to retaining the components of the maternal sample bound to the first stationary phase, the retained components of the maternal sample bound to the first stationary phase are contacted with the isolatably labeled affinity molecule. In some embodiments, the fetal cell-enriched sample preparation method further comprises: contacting the maternal sample with a second stationary phase having affinity for a fetal cell surface marker; separating components of the maternal sample bound to the second stationary phase from components of the maternal sample not bound to the second stationary phase; and retaining the components of the maternal sample bound to the second stationary phase. In some embodiments, the fetal cell surface marker is selected from the group consisting of MMP14 (matrix metalloproteinase 14), transferin receptor (CD71), glycophorin A (GPA), HLA-G, EGFR, thrombospondin receptor (CD36), CD 34, HbF, HAE 9, FB3-2, H3-3, erythropoietin receptor, HBE, AFP, APOC3, SERPINC1, AMBP, CPB2, ITIH1, APOH, HPX, beta-hCG, AHSG, APOB, J42-4-d, 2,3-biophosphoglycerate (BPG), Carbonic anhydrase (CA), and Thymidine kinase (TK). In some embodiments, the fetal cell surface marker is MMP14 (matrix metalloproteinase 14) or transferin receptor (CD71). In some embodiments, the maternal sample is a maternal blood sample. In some embodiments, at least 50%, 60%, 70%, 80%, or 90% of the cells in the fetal cell-enriched sample are fetal cells. In some embodiments, the fetal cell-enriched sample preparation method further comprises: providing the fetal cell-enriched sample; and analyzing a nucleotide sequence of a nucleic acid molecule or expression of a gene in two or more cells from the fetal cell-enriched sample. In some embodiments, the analyzing a nucleotide sequence of a nucleic acid molecule comprises sequencing genomic DNA of two or more cells from the fetal cell-enriched sample. In some embodiments, the sequencing genomic DNA comprises sequencing the DNA of a single cell, and wherein sequencing the DNA of a single cell is performed for two or more cells from the fetal cell-enriched sample. In some embodiments, sequencing the DNA of a single cell is performed for at least 2, 3, 4, 5, 6, 7, 8, 9 or 10 cells from the fetal cell-enriched sample. In some embodiments, the expression of a gene comprises hybridizing a detectable antibody to the surface of two or more cells from the fetal cell-enriched sample. In some embodiments, the analyzing a nucleotide sequence of a nucleic acid molecule comprises hybridizing a detectable probe to the genomic DNA of two or more cells from the fetal cell-enriched sample. In some embodiments, one or more individual cells are analyzed for hybridization of a detectable probe to the genomic DNA of each analyzed cell. In some embodiments, at least 2, 3, 4, 5, 6, 7, 8, 9 or 10 individual cells are analyzed for hybridization of a detectable probe to the genomic DNA of each analyzed cell.

### BRIEF DESCRIPTION OF THE FIGURES

FIG. 1 depicts an example of a protocol for obtaining enriched nucleated fetal red blood cells from a maternal blood sample.

FIG. 2 depicts an example of a protocol for assessing the nucleotide sequence of a nucleic acid molecule or expression of a gene determined for individual cells for each of two or more cells of a nucleated fetal red blood cell-enriched sample.

### DETAILED DESCRIPTION

The inventors have developed improved non-invasive fetal diagnostic methods. In particular, provided are methods of obtaining a fetal cell-enriched sample from a maternal sample and methods of assessing a maternal sample for a fetal nucleotide sequence or expression of a fetal gene.

This fetal material is a source of information about the gender and genetic makeup of the developing fetus. Fetal genetic material can be detected in maternal blood early in gestation. The methods provided herein include efficient protocols for isolating fetal cells from maternal blood with high purity. Some embodiments include an initial enrichment step that facilitates removal of many maternal non-nucleated cells through, for example, density gradient centrifugation. Subsequent purification or enrichment of fetal cells can include affinity-based separation. As an example, nucleated fetal erythroid precursor cells express a large number of galactose molecules on the cell surface, which can be captured by lectin. Thus, a lectin such as soybean agglutinin (SBA) can be used to enrich nucleated fetal cells using known methods such as those provided in Kitagawa et al., 2002 Prenat. Diagn 22:17-21. As another example, nucleated fetal cells have a variety of cell surface markers which can be used for further enrichment. In one such embodiment, matrix metalloproeteinase 14 (MMP14 or MMP-X1) precursor and/or transferin receptor (CD71) can be used to enrich fetal cells with both high specificity and a high recovery rate. Additional markers that can be used include, but are not limited to, glycophorin A (GPA), the thrombospondin receptor (CD36), CD 34, HbF, HAE 9, FB3-2, H3-3 and erythropoietin receptor. As an additional, optional step, negative selection using, for example, CD47, CD45, CD35, CD12, CD14, CD32 can be included, which can be used to specifically bind, and thereby remove, maternal erythrocytes.

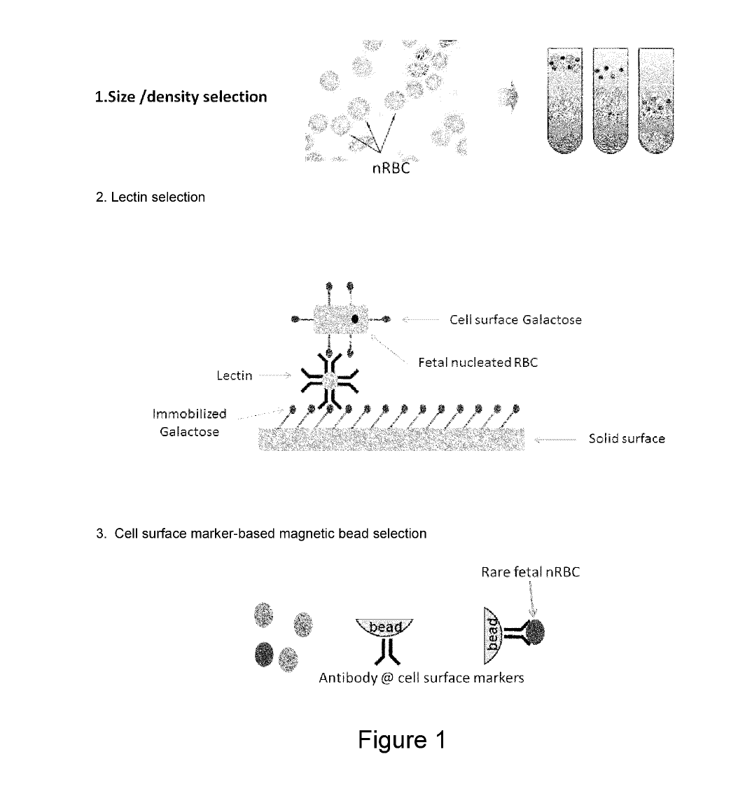

An example of a protocol for obtaining enriched nucleated fetal red blood cells from a maternal blood sample is provided in FIG. 1, which includes (1) size/density selection using density gradient centrifugation, (2) lectin selection using soybean agglutinin, and (3) cell surface marker-based magnetic bead section using, e.g., anti-MMP14 antibody.

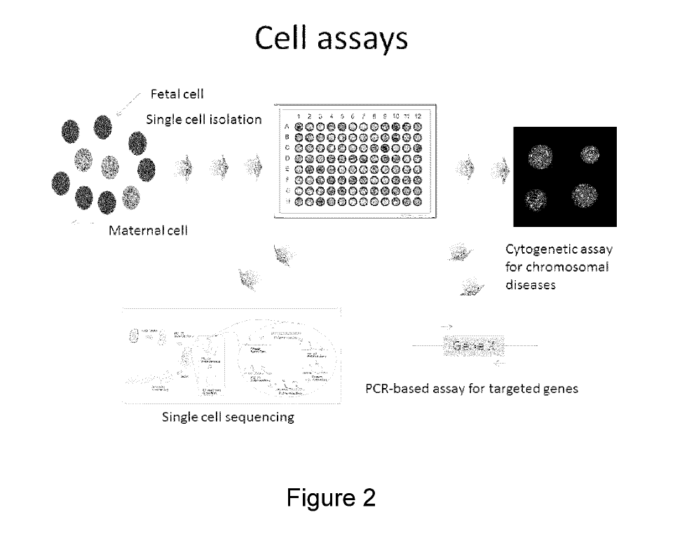

An example of a protocol for assessing the nucleotide sequence of a nucleic acid molecule or expression of a gene determined for individual cells for each of two or more cells of a nucleated fetal red blood cell-enriched sample is provided in FIG. 2, which includes (1) providing ˜10-100 cells from a nucleated fetal red blood cell-enriched sample, (2) separating the cells into 96 well plate (a well containing a cell is indicated by as a green well in the figure) such that each well contains 1 or 0 cells, and (3) the separated cells are subjected to assays that, for each individual well, determine the nucleotide sequence of a nucleic acid molecule or expression of a gene.

### Maternal Samples

Maternal Samples containing one or more nucleated fetal cells can be obtained from any animal for whom diagnosis or prognosis is to be performed or from an animal pregnant with a fetus for whom diagnosis or prognosis is to be performed. In one embodiment, a sample can be obtained from a female animal that is suspected of being pregnant, that is pregnant, or that has been pregnant. When the animal is a human, the sample can be taken during the first trimester (about the first three months of pregnancy), the 2nd trimester (about months 4-6 of pregnancy), or the third trimester (about months 7-9 of pregnancy). An animal of the present invention can be a human or a domesticated animal such as a cow, pig, horse, rabbit, dog, cat, sheep or goat. Typically, the sample obtained is a blood sample. Other samples can include vaginal discharge, cervical swab, and urine. Thus, for example, maternal samples, such as maternal blood samples can be obtained from a pregnant female human or non-human mammal and can be used to investigate the status of a fetus within the pregnant female human or non-human mammal.

When obtaining a maternal sample from an animal (e.g., blood sample), the amount of sample can vary depending upon animal size, its gestation period, and the condition being screened. In one embodiment, up to 200, 175, 150, 125, 100, 90, 80, 70, 60, 50, 40, 30, 20, 10, or 5 mL of a sample is obtained. In one embodiment, 5-200, 10-100, or 30-50 mL of sample is obtained. In one embodiment, more than 1, 2, 3, 4, 5, 10, 20, 30, 40, 50, 60, 70, 80, 90, 100 or 150 mL of a sample is obtained. In one embodiment between about 10-100 or 30-50 ml of a peripheral blood sample is obtained from a pregnant female. In some embodiments, a blood sample is obtained from a pregnant human or non-human animal within 36, 24, 22, 20, 18, 16, 14, 12, 10, 8, 6, or 4 weeks of conception or even after a pregnancy has terminated.

### Sample Enrichment/Purification

The sample is subjected to one or more steps that enrich the nucleated fetal cells relative to the total components of the sample and/or enrich the nucleated fetal cells relative to the total cells in the sample.

An example of a protocol for obtaining enriched nucleated fetal red blood cells from a maternal blood sample is provided in FIG. 1, which includes (1) size/density selection using density gradient centrifugation, (2) lectin selection using soybean agglutinin, and (3) cell surface marker-based magnetic bead section using, e.g., anti-MMP14 antibody. Variations of this example method can be performed in accordance with the teachings provided herein.

Density Gradient Centrifugation

Density gradient centrifugation is a method of separating cells based on the different densities of cell types in a mixture. The method can be used to separate cells into compartments which contain cells that are either lighter or heavier than a specific density of the gradient material(s) used. Density gradient centrifugation can be carried out through repetitive steps based on a series of different density gradients or in combination with other separation methods such as affinity separation, cell panning, cell sorting, and the like. In some embodiments, density gradient centrifugation can be performed using multiple layers of the different gradient densities. This method allows cells of different densities to form zones or bands at their corresponding densities after centrifugation. The cells in one or more different zones can be collected by placing a pipette at the appropriate location. Methods for enriching specific cell-types by density gradient centrifugation are described in U.S. Pat. No. 5,840,502, which is herein incorporated by reference in its entirety.

Methods of identifying fetal cells in a specimen using density gradient centrifugation utilize density gradient medium. The density gradient medium can be colloidal polyvinylpyrrolidone-coated silica (e.g. PercolD, Nycodenz), a nonionic polysucrose (Ficoll) either alone or with sodium diatrizoate (e.g. Ficoll-Paque or Histopaque), or mixtures thereof. The density of the reagent employed is selected to separate the nucleated fetal cells of interest from other blood components, such as non-cellular components and non-nucleated red blood cells.

Size-Based Enrichment

In some embodiments, enrichment of rare cells occurs using one or more size-based separation methods. Examples of size-based separation modules include filtration membranes, molecular sieves, and matrixes. Examples of size-based separation modules contemplated by the present invention include those disclosed in International Publication No. WO 2004/113877, which is herein incorporated by reference in its entirety. Other size based separation methods are disclosed in International Publication No. WO 2004/0144651 and U.S. Patent Application Publication Nos. US20080138809A1 and US20080220422A1, which are herein incorporated by reference in their entirety.

Affinity-Based Enrichment

In some embodiments, nucleated fetal cells can be enriched based on their affinity for a binding moiety or affinity molecule. In such embodiments, the binding moiety is isolatably labeled to facilitate separation of the nucleated fetal cells from undesired components of a maternal sample. For example, a binding moiety with affinity for a nucleated fetal cell can bind the nucleated fetal cell and can be used to separate nucleated fetal cells by being bound to a solid support such as a magnetic bead or the solid phase of a chromatographic material, or the binding moiety can be detectably labeled such that the nucleated fetal cells can be distinguished from other sample components by detection-assisted enrichment of the nucleated fetal cells.

In some embodiments, the affinity methods include using an isolatably labeled binding moiety or affinity molecule having affinity for a fetal cell surface marker. For example, a binding moiety or affinity molecule can be attached to a stationary phase, a fluorophore, a radionuclide, or other detectable moiety, and the sample can be contacted with the isolatably labeled binding moiety or affinity molecule under conditions that allow the fetal nucleated cells to be specifically bound to the binding moiety or affinity molecule while other components of the sample do not specifically bind to the binding moiety or affinity molecule. The contacted isolatably labeled binding moiety or affinity molecule can then be treated, for example using optical tweezers, magnetic stand, density centrifuge, flow cytometry, and size-based liquid chromatography, to separate components of the maternal sample bound to the isolatably labeled binding moiety or affinity molecule from components of the maternal sample not bound to the isolatably labeled binding moiety or affinity molecule. The bound components of the maternal sample can optionally be washed to remove non-specifically bound components. The components of the sample bound to the isolatably labeled binding moiety or affinity molecule, which includes fetal nucleated cells, can then be retained or harvested for further enrichment or for analysis.

Binding moieties can include e.g., proteins, nucleic acids, and carbohydrates that specifically bind to nucleated fetal cells. In one embodiment, the binding moiety has affinity for one or more carbohydrates, such as galactose. For example, a binding moiety can be lectin. In other embodiments, the binding moiety is an antibody. Examples of such binding moiety antibodies include: anti-matrix metalloproteinase 14 (anti-MMP14), anti-transferin receptor (anti-CD71), anti-glycophorin A (anti-GPA), anti-thrombospondin receptor (anti-CD36), anti-CD34, anti-HbF, anti-HAE9, anti-FB3-2, anti-H3-3, anti-erythropoietin receptor, anti-CD235a, anti-carbohydrates, anti-selectin, anti-CD45, anti-GPA, anti-antigen-i, anti-EpCAM, anti-E-cadherin, anti-Muc-1, anti-hPL, anti-CHS2, anti-KISS1, anti-GDF15, anti-CRH, anti-TFP12, anti-CGB, anti-LOC90625, anti-FN1, anti-COL1A2, anti-PSG9, anti-PSG1, anti-HBE, anti-AFP, anti-APOC3, anti-SERPINC1, anti-AMBP, anti-CPB2, anti-ITIH1, anti-APOH, anti-HPX, anti-beta-hCG, anti-AHSG, anti-APOB, anti-J42-4-d, anti-2,3-biophosphoglycerate (anti-BPG), anti-Carbonic anhydrase (anti-CA), or anti-Thymidine kinase (anti-TK).

In one embodiment, a nucleated fetal cell is enriched using anti-MMP14, anti-CD71 and/or anti-GPA selection. In another embodiment, a trophoblast is enriched using anti-HLA-G or anti-EGFR selection. In another embodiment, nucleated fetal cells are enriched using one or more antibodies or antibody fragments that can bind a protein expressed from the genes MMP14, CD71, GPA, HLA-G, EGFR, CD36, CD34, HbF, HAE 9, FB3-2, H3-3, erythropoietin receptor, HBE, AFP, APOC3, SERPINC1, AMBP, CPB2, ITIH1, APOH, HPX, beta-hCG, AHSG, APOB, J42-4-d, BPG, CA, or TK.

Stationary Phase-Based Enrichment

In some embodiments, affinity chromatographic methods can be used. For example, a binding moiety or affinity molecule can be attached to a stationary phase, such as a bead, column or particle, and the sample can be contacted with the affinity molecule-attached stationary phase under conditions that allow the fetal nucleated cells to be specifically bound to the binding moiety or affinity molecule while other components of the sample do not specifically bind to the binding moiety or affinity molecule. The contacted stationary phase can then be treated, for example, using a mobile phase, to separate components of the maternal sample bound to the affinity molecule-attached stationary phase from components of the maternal sample not bound to the affinity molecule-attached stationary phase. The components of the sample bound to the affinity molecule-attached stationary phase, which includes fetal nucleated cells, can then be retained or harvested for further enrichment or for analysis.

In one embodiment, a magnetic particle is used to enrich nucleated fetal cells. In one embodiment, a binding moiety such as an antibody can be coupled to a magnetic particle (e.g., a magnetic bead). In one embodiment the bead is couple to an antibody or fragment of an antibody that is an anti-MMP14, anti-CD71, anti-GPA, anti-CD36, anti-CD34, anti-HbF, anti-HAE9, anti-FB3-2, anti-H3-3, anti-erythropoietin receptor, anti-CD235a, anti-carbohydrates, anti-selectin, anti-CD45, anti-GPA, anti-antigen-i, anti-EpCAM, anti-E-cadherin, anti-Muc-1, anti-hPL, anti-CHS2, anti-KISS1, anti-GDF15, anti-CRH, anti-TFP12, anti-CGB, anti-LOC90625, anti-FN1, anti-COL1A2, anti-PSG9, anti-PSG1, anti-HBE, anti-AFP, anti-APOC3, anti-SERPINC1, anti-AMBP, anti-CPB2, anti-ITIH1, anti-APOH, anti-HPX, anti-beta-hCG, anti-AHSG, anti-APOB, anti-J42-4-d, anti-BPG, anti-CA, or anti-TK antibody or fragment of an antibody.

Efficiency of Enrichment

In some embodiments, even an enriched product can be dominated (>50%) by cells not of interest (e.g., nucleated maternal red blood cells). In some cases, the nucleated fetal cells of an enriched sample makes up at least 2, 3, 4, 5, 10, 20, 30, 40, 50, 60, 70, 80, 90 or 95% of all cells in the enriched sample. For example, using the methods and systems described herein, a maternal blood sample of 20 mL from a pregnant human can be enriched for one or more nucleated fetal cells, such as nucleated red blood cells, such that the enriched sample has a total of about 500 cells, 2% of which are nucleated fetal cells and the rest of the cells are maternal. In some embodiments, the enrichment steps performed have removed at least 50, 60, 70, 80, 85, 90, 91, 92, 93, 94, 95, 96, 97, 98, 99, 99.5, 99.6, 99.7, 99.8 or 99.9% of all unwanted analytes (e.g., maternal cells, maternal red blood cells, nucleated maternal red blood cells, anucleate cells) from a sample.

### Fetal Biomarkers

In some embodiments fetal biomarkers can be used to detect and/or isolate one or more fetal cells. For example, this can be performed by distinguishing between fetal and maternal nucleated cells based on relative expression of a gene (e.g., DYS1, DYZ, CD-71, MMP14) that is differentially expressed during fetal development. In one embodiment of the provided invention, detection of transcript or protein expression of one or more genes including, MMP14, CD71, GPA, HLA-G, EGFR, CD36, CD34, HbF, HAE 9, FB3-2, H3-3, erythropoietin receptor, HBE, AFP, APOC3, SERPINC1, AMBP, CPB2, ITIH1, APOH, HPX, beta-hCG, AHSG, APOB, J42-4-d, 2,3-biophosphoglycerate (BPG), Carbonic anhydrase (CA), or Thymidine kinase (TK), is used to enrich, purify, enumerate, identify detect or distinguish a fetal cell. The expression can include a transcript expressed from these genes or a protein. In one embodiment of the provided invention, expression of one or more genes including MMP14, CD71, GPA, HLA-G, EGFR, CD36, CD34, HbF, HAE 9, FB3-2, H3-3, erythropoietin receptor, HBE, AFP, AHSG, J42-4-d, BPG, CA, or TK, is used to identify, purify, enrich, or enumerate a nucleated fetal cell such as a nucleated fetal red blood cell.

Beta-hCG (also known as b-hCG, HCG, CGB, CGB3 and hCGB) is a member of the glycoprotein hormone beta chain family and encodes the beta 3 subunit of chorionic gonadotropin (CG). Glycoprotein hormones are heterodimers consisting of a common alpha subunit and an unique beta subunit which confers biological specificity. CG is produced by the trophoblastic cells of the placenta and stimulates the ovaries to synthesize the steroids that are essential for the maintenance of pregnancy. The beta subunit of CG is encoded by 6 genes which are arranged in tandem and inverted pairs on chromosome 19q13.3 and contiguous with the luteinizing hormone beta subunit gene.

APOB (also known as apolipoprotein B (including Ag(x) antigen) and FLDB) is the main apolipoprotein of chylomicrons and low density lipoproteins. It occurs in plasma as two main isoforms, apoB-48 and apoB-100: the former is synthesized exclusively in the gut and the latter in the liver. The intestinal and the hepatic forms of apoB are encoded by a single gene from a single, very long mRNA. The two isoforms share a common N-terminal sequence. The shorter apoB-48 protein is produced after RNA editing of the apoB-100 transcript at residue 2180 (CAA->UAA), resulting in the creation of a stop codon, and early translation termination. Mutations in this gene or its regulatory region cause hypobetalipoproteinemia, normotriglyceridemic hypobetalipoproteinemia, and hypercholesterolemia due to ligand-defective apoB, diseases affecting plasma cholesterol and apoB levels.

AHSG (also known as alpha-2-HS-glycoprotein; AHS; A2HS; HSGA; and FETUA) is a glycoprotein present in the serum and can be synthesized by hepatocytes. The AHSG molecule consists of two polypeptide chains, which are both cleaved from a proprotein encoded from a single mRNA. It is involved in several functions, such as endocytosis, brain development and the formation of bone tissue. The protein is commonly present in the cortical plate of the immature cerebral cortex and bone marrow hemopoietic matrix, and it has therefore been postulated that it participates in the development of the tissues.

HPX (also known as hemopexin) can bind heme. It can protect the body from the oxidative damage that can be caused by free heme by scavenging the heme released or lost by the turnover of heme proteins such as hemoglobin. To preserve the body's iron, upon interacting with a specific receptor situated on the surface of liver cells, hemopexin can release its bound ligand for internalisation.

CPB2 (also known as carboxypeptidase B2 (plasma); CPU; PCPB; and TAFI) is an enzyme that can hydrolyze C-terminal peptide bonds. The carboxypeptidase family includes metallo-, serine, and cysteine carboxypeptidases. According to their substrate specificity, these enzymes are referred to as carboxypeptidase A (cleaving aliphatic residues) or carboxypeptidase B (cleaving basic amino residues). The protein encoded by this gene is activated by trypsin and acts on carboxypeptidase B substrates. After thrombin activation, the mature protein downregulates fibrinolysis. Polymorphisms have been described for this gene and its promoter region. Available sequence data analyses indicate splice variants that encode different isoforms.

ITIH1 (also known as inter-alpha (globulin) inhibitor H1; H1P; ITIH; LATIH; and MGC126415) is a serine protease inhibitor family member. It is assembled from two precursor proteins: a light chain and either one or two heavy chains. ITIH1 can increase cell attachment in vitro.

APOH (also known as apolipoprotein H (beta-2-glycoprotein I); BG; and B2G1) has been implicated in a variety of physiologic pathways including lipoprotein metabolism, coagulation, and the production of antiphospholipid autoantibodies. APOH may be a required cofactor for anionic phospholipid binding by the antiphospholipid autoantibodies found in sera of many patients with lupus and primary antiphospholipid syndrome.

AMBP (also known as alpha-1-microglobulin/bikunin precursor; HCP; ITI; UTI; EDC1; HI30; ITIL; IATIL; and ITILC) encodes a complex glycoprotein secreted in plasma. The precursor is proteolytically processed into distinct functioning proteins: alpha-1-microglobulin, which belongs to the superfamily of lipocalin transport proteins and may play a role in the regulation of inflammatory processes, and bikunin, which is a urinary trypsin inhibitor belonging to the superfamily of Kunitz-type protease inhibitors and plays an important role in many physiological and pathological processes. This gene is located on chromosome 9 in a cluster of lipocalin genes.

J42-4-d is also known as t-complex 11 (mouse)-like 2; MGC40368 and TCP11L2.

Assessing the Enriched Cells

Cells from the fetal cell-enriched sample can then be assessed for a nucleotide sequence of a nucleic acid molecule or expression of a gene.

An example of a protocol for assessing the nucleotide sequence of a nucleic acid molecule or expression of a gene determined for individual cells for each of two or more cells of a nucleated fetal red blood cell-enriched sample is provided in FIG. 2, which includes (1) providing ˜10-100 cells from a nucleated fetal red blood cell-enriched sample, (2) separating the cells into 96 well plate (a well containing a cell is indicated by as a green well in the figure) such that each well contains 1 or 0 cells, and (3) the separated cells are subjected to assays that, for each individual well, determine the nucleotide sequence of a nucleic acid molecule or expression of a gene. Variations of this example method can be made in accordance with the teachings provided herein.

Cell Assays

Enriched nucleated fetal cells can be analyzed by one or more cell assays that identify one or more nucleotide sequences of the nucleated fetal cell or expression of one or more genes of the nucleated fetal cell.

In some embodiments, the expression of one or more genes of the nucleated fetal cell is assessed for two or more cells from the fetal cell-enriched sample. In some embodiments, the expression of one or more genes of the nucleated fetal cell comprises gene expression for a single cell. In some embodiments, expression of one or more genes of a single cell is assessed for two or more cells from the fetal cell-enriched sample. In some embodiments, expression of one or more genes of a single cell is assessed for at least 2, 3, 4, 5, 6, 7, 8, 9 or 10 cells from the fetal cell-enriched sample.

In some embodiments, the assessing a nucleotide sequence of a nucleic acid molecule comprises sequencing genomic DNA in a cell from the fetal cell-enriched sample. In some embodiments, analyzing a nucleotide sequence of a nucleic acid molecule comprises hybridizing a detectable probe that hybridizes to a sequence of interest. In some embodiments, the nucleotide sequence(s) of the nucleated fetal cell is assessed for two or more cells from the fetal cell-enriched sample. In some embodiments, the sequencing genomic DNA comprises sequencing the DNA of a single cell. In some embodiments, sequencing the DNA of a single cell is performed for two or more cells from the fetal cell-enriched sample. In some embodiments, sequencing the DNA of a single cell is performed for at least 2, 3, 4, 5, 6, 7, 8, 9 or 10 cells from the fetal cell-enriched sample. In some embodiments, the analyzing a nucleotide sequence of a nucleic acid molecule comprises hybridizing a detectable probe to the genomic DNA of two or more cells from the fetal cell-enriched sample. In some embodiments, one or more individual cells are analyzed for hybridization of a detectable probe to the genomic DNA of each analyzed cell. In some embodiments, at least 2, 3, 4, 5, 6, 7, 8, 9 or 10 individual cells are analyzed for hybridization of a detectable probe to the genomic DNA of each analyzed cell.

In some embodiments, gene expression is assessed by detecting RNA expression of a gene in a cell from the fetal cell-enriched sample. In some embodiments, gene expression is assessed by detecting protein expression of a gene in a cell from the fetal cell-enriched sample. In some embodiments, the expression of a gene comprises hybridizing a detectable antibody to the surface of a cell from the fetal cell-enriched sample.

Nucleotide sequencing methods include targeted nucleotide sequencing methods and genomic nucleotide sequencing methods. Targeted sequencing methods include sequencing of one or more particular genes or other targets using, for example, primer-based PCR to amplify the targeted nucleic acid region, followed by routine nucleotide sequencing as known in the art. Genomic nucleotide sequencing methods include methods for amplification and nucleotide sequencing of genomic DNA. Genomic nucleotide sequencing methods include, but are not limited to multiple displacement amplification in single cells, as taught by Zhang et al. (2006) “Sequencing genomes from single cells by polymerase cloning” Nature Biotechnology 24(6):680-6; and Spits et al. (2006) “Whole-genome multiple displacement amplification from single cells” Nature protocols 1(4):1965-70. Further genomic nucleotide sequencing methods include, but are not limited to multiple displacement amplification in single cells, as taught by multiple annealing and looping-based amplification cycles (MALBAC), as taught by Zong et al. (2012) “Genome-Wide Detection of Single-Nucleotide and Copy-Number Variations of a Single Human Cell” Science 338:1622-6. In some embodiments, after whole genome amplification, partial genome sequences of interest can be sequenced after hybridization-based selection with user-designed oligos (such as whole exome sequencing).

Expression of a gene can be determined by, for example, detecting transcript or protein expressed from a gene. Expression of a transcript from a gene can be detected by, for example, RNA chromogenic in situ hybridization (CISH), RNA FISH, RNA-FISH using a molecular beacon probe, Q-PCR, RT-PCR, Taqman RT-PCR, Northern blotting, ribonuclease protection assay, RNA expression profiling using microarrays or whole transcriptome sequencing.

Protein expression can be detected by, e.g., immunohistochemistry, immunocytochemistry, Western blotting, mass spectrometry, ELISA, gel electrophoresis followed by Coomassie staining or silver staining, flow cytometry, FACS, or microfluidic fluorescent cell sorting. The expressed protein can be a cell surface or an internal expressed protein. The cell surface protein can be recognized by a binding moiety, e.g., an antibody based moiety. The binding moieties used in detection can be an antibody, Fab fragment, Fc fragment, scFv fragment, peptidomimetic, or peptoid.

In one embodiment, the expression levels are determined by measuring nuclear RNA transcripts including, nascent or unprocessed transcripts. In another embodiment, expression levels are determined by measuring mRNA, including ribosomal RNA. There are many methods known in the art for imaging (e.g., measuring) nucleic acids or RNA including, but not limited to, using expression arrays from Affymetrix, Inc. or sequencing from Illumina, Inc. and Life Technologies, Inc.

RT-PCR primers can be designed by targeting gene-specific regions, selecting the amplicon size, and adjusting the primers annealing temperature to achieve equal PCR amplification efficiency. Thus TaqMan probes can be designed for each of the amplicons with well-separated fluorescent dyes, Alexa Fluor-355, Alexa Fluor-488, and Alexa Fluor-555. The primers selected can be validated first in a duplex format to verify their specificity, limit of detection, and amplification efficiency using target cDNA templates. The best combinations of primers can be further tested in a triplex format for its amplification efficiency, detection dynamic range, and limit of detection.

Various commercially available reagents are available for RT-PCR, such as One-step RT-PCR reagents, including Qiagen One-Step RT-PCR Kit and Applied Biosystems TaqMan One-Step RT-PCR Master Mix Reagents kit. Forward primers can be labeled for each of the targets. Enriched cells can be deposited by cytospinning onto glass slides. Additionally, cytospinning the enriched cells can be performed after in situ RT-PCR. Thereafter, the presence of the fluorescent-labeled amplicons can be visualized by fluorescence microscopy. The reverse transcription time and PCR cycles can be optimized to maximize the amplicon signal:background ratio to have maximal separation of fetal over maternal signature. In some embodiments, signal:background ratio is greater than 5, 10, 50, or 100 and the overall cell loss during the process is less than 50, 10 or 5%.

Any of a variety of fluorescent molecules or dyes that can be used with the nucleic acid, antibody or antibody-based fragment probes provided herein, including, but not limited to, Alexa Fluor 350, AMCA, Alexa Fluor 488, Fluorescein isothiocyanate (FITC), GFP, RFP, YFP, BFP, CFSE, CFDA-SE, DyLight 288, SpectrumGreen, Alexa Fluor 532, Rhodamine, Rhodamine 6G, Alexa Fluor 546, Cy3 dye, tetramethylrhodamine (TRITC), SpectrumOrange, Alexa Fluor 555, Alexa Fluor 568, Lissamine rhodamine B dye, Alexa Fluor 594, Texas Red dye, SpectrumRed, Alexa Fluor 647, Cy5 dye, Alexa Fluor 660, Cy5.5 dye, Alexa Fluor 680, Phycoerythrin (PE), Propidium iodide (PI), Peridinin chlorophyll protein (PerCP), PE-Alexa Fluor 700, PE-Cy5 (TRI-COLOR), PE-Alexa Fluor 750, PE-Cy7, APC, APC-Cy7, Draq-5, Pacific Orange, Amine Aqua, Pacific Blue, Alexa Fluor 405, Alexa Fluor 430, Alexa Fluor 500, Alexa Fluor 514, Alexa Fluor-555, Alexa fluor-568, Alexa Fluor-610, Alexa Fluor-633, DyLight 405, DyLight 488, DyLight 549, DyLight 594, DyLight 633, DyLight 649, DyLight 680, DyLight 750, or DyLight 800.

In one embodiment the presence of or transcript expression of one or more genes in a fetal cell can be detected using one or more primer/probe sets. For example, at least 1, 2, 3, 4, 5, 6 or more primer/probe sets can be used to detect expression of one or more genes in a fetal cell (e.g., a nucleated fetal red blood cell). In one embodiment a primer/probe set comprises two primers and one probe and optionally a quencher. In one embodiment a multiplex primer/probe combination comprises one or more primer/probe sets. In one embodiment a primer/probe set or a multiplex primer/probe combination is combined with a sample for q-PCR. In one embodiment a primer/probe set or a multiplex primer/probe combination is combined with a sample for Real Time-PCR. In one embodiment a multiplex primer/probe combination can be designed so as to balance the amounts of the primers and probes for each set so that a detectable signal is produced for each primer/probe set, if a target sequence is present in a sample. In one embodiment optimum annealing temperatures and thermocycling profiles can be designed so that multiple primer/probe combination can function in the same reaction chamber to detect the presence of a target sequence in sample. In one embodiment the probes are labeled with different fluorescent dyes. The dye labeled probes can be optimized so that each probe from a particular primer/probe set in a multiplex reaction, is labeled with a different dye that fluoresces at a peak wavelength sufficiently different from the other dye labeled probes so as to allow identification of the fluorescence from each sets probe. In one embodiment, a multiplex primer/probe combination comprises one or more primer/probe sets that anneal to a genomic DNA, of the MMP14, CD71, GPA, HLA-G, EGFR, CD36, CD34, HbF, HAE 9, FB3-2, H3-3, erythropoietin receptor, HBE, AFP, APOC3, SERPINC1, AMBP, CPB2, ITIH1, APOH, HPX, beta-hCG, AHSG, APOB, J42-4-d, BPG, CA, or TK genes. In another embodiment, a multiplex primer/probe combination comprises one or more primer/probe sets that anneal to a RNA expressed by, or a cDNA of an RNA expressed by the MMP14, CD71, GPA, HLA-G, EGFR, CD36, CD34, HbF, HAE 9, FB3-2, H3-3, erythropoietin receptor, HBE, AFP, APOC3, SERPINC1, AMBP, CPB2, ITIH1, APOH, HPX, beta-hCG, AHSG, APOB, J42-4-d, BPG, CA, or TK genes. In one embodiment, a nucleated fetal cell is enriched, enumerated, purified, detected or identified using a multiplex primer/probe combination comprising one or more primer/probe sets that anneal to a genomic DNA, a RNA expressed by, or a cDNA of an RNA expressed by the MMP14, CD71, GPA, HLA-G, EGFR, CD36, CD34, HbF, HAE 9, FB3-2, H3-3, erythropoietin receptor, HBE, AFP, APOC3, SERPINC1, AMBP, CPB2, ITIH1, APOH, HPX, beta-hCG, AHSG, APOB, J42-4-d, BPG, CA, or TK genes. In another embodiment a multiplex primer/probe combination comprises at least three primer/probe sets that anneal to a genomic DNA, a RNA expressed by, or a cDNA of an RNA expressed by the FN1, beta-hCG, or AHSG genes.

In another embodiment at least 1, 2, 3, 4, 5, 6 or more sets of primers can be used to detect the presence of or transcript expression of one or more genes in a nucleated fetal cell. In one embodiment a primer set comprises two primers. In one embodiment two or more primer sets are included in a multiplex reaction with a sample, comprising a target sequence. In one embodiment a multiplex primer combination can be designed so as to balance the amounts of the primers for each set so that a detectable amplified product is produced for each primer set, if a target sequence is present in a sample. In one embodiment optimum annealing temperatures and thermocycling profiles can be designed so that multiple primer sets can be combined to function in the same reaction chamber to amplify the presence of a target sequence in sample. In one embodiment, a primer set anneals to a genomic DNA, of the MMP14, CD71, GPA, HLA-G, EGFR, CD36, CD34, HbF, HAE 9, FB3-2, H3-3, erythropoietin receptor, HBE, AFP, APOC3, SERPINC1, AMBP, CPB2, ITIH1, APOH, HPX, beta-hCG, AHSG, APOB, J42-4-d, BPG, CA, or TK genes. In another embodiment, a primer set anneals to an RNA expressed by, or a cDNA of an RNA expressed by the MMP14, CD71, GPA, HLA-G, EGFR, CD36, CD34, HbF, HAE 9, FB3-2, H3-3, erythropoietin receptor, HBE, AFP, APOC3, SERPINC1, AMBP, CPB2, ITIH1, APOH, HPX, beta-hCG, AHSG, APOB, J42-4-d, BPG, CA, or TK genes. In one embodiment, a nucleated fetal cell is enriched, enumerated, purified, detected or identified using a primer set that anneals to a genomic DNA, a RNA expressed by, or a cDNA of an RNA expressed by the MMP14, CD71, GPA, HLA-G, EGFR, CD36, CD34, HbF, HAE 9, FB3-2, H3-3, erythropoietin receptor, HBE, AFP, APOC3, SERPINC1, AMBP, CPB2, ITIH1, APOH, HPX, beta-hCG, AHSG, APOB, J42-4-d, BPG, CA, or TK genes.

In another embodiment at least 1, 2, 3, 4, 5, 6 or more probes can be used to detect transcript expression of one or more genes by a nucleated fetal cell. In one embodiment two or more probes are detectably labeled and can bind to an RNA sequence. In one embodiment two or more probes are used to detect more than one RNA sequence expressed by a fetal cell. In one embodiment the two or more probes are used in a method of fluorescent in-situ hybridization. In one embodiment the method of fluorescent in-situ hybridization is RNA-FISH. In one embodiment the probes are nucleic acid probes. In another embodiment the probe is a peptide nucleic acid (PNA). In another embodiment a probe comprises one or more modified nucleic acids, such as an amide modified nucleic acid, a phosphoramidate modified nucleic acid, a boranophosphate modified nucleic acid, a methylphosophonate modified nucleic acid, a deoxyribonucleic guanidine (DNG) modified nucleic acid or a morpholino modified nucleic acid.

In one embodiment two or more probes are labeled with a detectable tag, such as biotin or streptavidin, which can bind to a labeled conjugate. In another embodiment the probe is labeled with an enzyme (such as alkaline phosphatase) that can convert a substrate (such as Fast Red) into a detectable label. In one embodiment the enzyme is alkaline phosphatase, horseradish peroxidase, beta-galactosidase, or glucose oxidase.

In one embodiment the conjugate is labeled with a fluorescent dye. In another embodiment, two or more probes are detectably labeled by fluorescent labeling. In one embodiment two or more probes are labeled with the same fluorescent label. In one embodiment two or more probes are labeled with different fluorescent labels. The fluorescently labeled probes can be optimized so that each probe from is labeled with a different label that fluoresces at a peak wavelength sufficiently different from the other fluorescently labeled probe so as to allow identification of the fluorescence from each probe.

In one embodiment, one or more detectably labeled probes anneal to an RNA sequence or specifically bind to a polypeptide expressed by an MMP14, CD71, GPA, HLA-G, EGFR, CD36, CD34, HbF, HAE 9, FB3-2, H3-3, erythropoietin receptor, HBE, AFP, APOC3, SERPINC1, AMBP, CPB2, ITIH1, APOH, HPX, beta-hCG, AHSG, APOB, J42-4-d, BPG, CA, or TK gene. In one embodiment, a nucleated fetal cell is enriched, enumerated, purified, detected or identified using one or more detectably labeled probes that anneal to an RNA sequence or specifically bind to a polypeptide expressed by one or more of the MMP14, CD71, GPA, HLA-G, EGFR, CD36, CD34, HbF, HAE 9, FB3-2, H3-3, erythropoietin receptor, HBE, AFP, APOC3, SERPINC1, AMBP, CPB2, ITIH1, APOH, HPX, beta-hCG, AHSG, APOB, J42-4-d, BPG, CA, or TK genes.

In another embodiment at least 1, 2, 3, 4, 5, 6 or more antibodies or antibody-based fragments can be used to detect expression of one or more proteins in a fetal cell (e.g., a fnRBC or trophoblast).

In one embodiment an antibody or antibody-based fragment is labeled with a detectable tag, such as biotin or streptavidin, which can bind to a labeled conjugate. In another embodiment an antibody or antibody-based fragment is labeled with an enzyme (such as alkaline phosphatase) that can convert a substrate (such as Fast Red) into a detectable label. In one embodiment the enzyme is alkaline phosphatase, horseradish peroxidase, beta-galactosidase, or glucose oxidase.

In one embodiment an antibody or antibody fragment binds to a fetal cell marker protein. In one embodiment an antibody or antibody fragment is labeled with a fluorescent dye. In another embodiment an antibody or antibody fragment binds to an antibody or antibody fragment labeled with a fluorescent dye. In one embodiment more than one antibody or antibody fragments is labeled with the same fluorescent dye. In one embodiment each antibody or antibody fragment is labeled with a different fluorescent dye. The dye labeled antibody or antibody-based fragment can be optimized so that each antibody or antibody-based fragment is labeled with a different dye that fluoresces at a peak wavelength sufficiently different from another dye labeled antibody or antibody-based fragment so as to allow identification of the fluorescence from each antibody or antibody-based fragment.

In one embodiment at least 1, 2, 3, 4, 5, 6 or more anti-MMP14, anti-CD71, anti-GPA, anti-CD36, anti-CD34, anti-HbF, anti-HAE9, anti-FB3-2, anti-H3-3, anti-erythropoietin receptor, anti-CD235a, anti-carbohydrates, anti-selectin, anti-CD45, anti-GPA, anti-antigen-i, anti-EpCAM, anti-E-cadherin, anti-Muc-1, anti-hPL, anti-CHS2, anti-KISS1, anti-GDF15, anti-CRH, anti-TFP12, anti-CGB, anti-LOC90625, anti-FN1, anti-COL1A2, anti-PSG9, anti-PSG1, anti-HBE, anti-AFP, anti-APOC3, anti-SERPINC1, anti-AMBP, anti-CPB2, anti-ITIH1, anti-APOH, anti-HPX, anti-beta-hCG, anti-AHSG, anti-APOB, anti-J42-4-d, anti-BPG, anti-CA, or anti-TK antibodies or antibody-based fragments are used to detect expression of one or more proteins by a fetal cell. In one embodiment an antibody or antibody-based fragment binds to a protein within a fetal cell. In another embodiment an antibody, or antibody-based fragment binds to a protein expressed on the surface of a fetal cell.

The detection of protein or transcript expression by specific genes can be used to distinguish a fetal cell from a reference cell, e.g., a maternal cell, distinguish between fetal cell types, identify a fetal cell, purify or enrich one or more fetal cells, or for enumeration of one or more fetal cells.

In one embodiment, cell type specific fetal cell markers can be used to identify the fetal cell types by an RT-PCR approach.

In one embodiment, a fetal cell can be labeled by RNA FISH. In one embodiment a fetal cell can be labeled with a molecular beacon. In one embodiment a fetal cell labeled with a molecular beacon can be identified, purified, enriched or enumerated by FACS or microfluidic fluorescent cell sorting.

In one embodiment, by combining RT-PCR and digital PCR, fetal cell types can be identified and the fetal cell numbers counted.

In one embodiment, a fetal cell can be labeled by an antibody or antibody-based fragment that binds to a protein expressed by a fetal cell marker gene. In one embodiment a fetal cell labeled with an antibody or antibody-based fragment can be identified, purified, enriched or enumerated by FACS or microfluidic fluorescent cell sorting.

Chromosomal analysis also can be performed using one or more cytogenetic methods known in the art or otherwise provided herein, including, but not limited to, routine analysis of G-banded chromosomes, other cytogenetic banding techniques, as well as molecular cytogenetics such as fluorescent in situ hybridization (FISH) and comparative genomic hybridization (CGH).

In one embodiment, sample analysis involves performing one or more genetic analyses or detection steps on nucleic acids from the fetal cell-enriched sample. Nucleic acids from enriched cells or enriched nuclei that can be analyzed by the methods herein include: double-stranded DNA, single-stranded DNA, single-stranded DNA hairpins, DNA/RNA hybrids, RNA (e.g. mRNA) and RNA hairpins. Examples of genetic analyses that can be performed on enriched cells or nucleic acids include, e.g., SNP detection, STR detection, and RNA expression analysis.

In one embodiment, analysis involves detecting one or more mutations or SNPs in DNA or RNA transcripts from one or more cells of the fetal cell-enriched sample. Such detection can be performed using, for example, DNA microarrays or RNA transcript expression arrays. Examples of DNA microarrays include those commercially available from Affymetrix, Inc. (Santa Clara, Calif.) according to methods known in the art, as demonstrated by Kennedy, G. C., et al., Nature Biotechnology 21, 1233-1237, 2003; Liu, W. M., Bioinformatics 19, 2397-2403, 2003; Matsuzaki, H., Genome Research 3, 414-25, 2004; and Matsuzaki, H., Nature Methods, 1, 109-111, 2004 as well as in U.S. Pat. Nos. 5,445,934; 5,744,305; 6,261,776; 6,291,183; 5,799,637; 5,945,334; 6,346,413; 6,399,365; and 6,610,482, and EP 619 321; 373 203, which are herein incorporated by reference in their entirety. In one embodiment, a microarray is used to detect at least 5, 10, 20, 50, 100, 200, 500, 1,000, 2,000, 5,000 10,000, 20,000, 50,000, 100,000, 200,000, or 500,000 different nucleic acid target(s) in a sample.

A computer program comprising a computer readable medium having a computer executable logic can be used to automate genotyping clusters and callings.

In any of the embodiments herein, genotyping (e.g., SNP detection) and/or expression analysis (e.g., RNA transcript quantification) of genetic content from enriched rare cells or enriched rare cell nuclei can be accomplished by sequencing. Sequencing can be accomplished through classic Sanger sequencing methods which are well known in the art. Sequencing can also be accomplished using high-throughput systems some of which allow detection of a sequenced nucleotide immediately after or upon its incorporation into a growing strand, i.e., detection of sequence in real time or substantially real time. In one embodiment, high throughput sequencing generates at least 10,000, at least 50,000, at least 100,000, at least 200,000, at least 300,000, at least 400,000, at least 500,000, at least 1,000,000 or at least 5,000,000 sequence reads per hour; with each read being at least 50, at least 60, at least 70, at least 80, at least 90, at least 100, at least 120 or at least 150 bases per read. Sequencing can be performed using genomic DNA or cDNA derived from RNA transcripts as a template. In one embodiment, high-throughput sequencing can be used, including high-throughput sequencing of a single cell from the fetal cell-enriched sample. In one embodiment, cDNAs, which are reverse transcribed from mRNAs obtained from fetal or maternal cells, are analyzed. The type and abundance of the cDNAs can be used to determine whether a cell is a fetal cell (such as by the presence of Y chromosome specific transcripts) or whether the fetal cell has a genetic abnormality (such as aneuploidy, abundance or type of alternative transcripts or problems with DNA methylation or imprinting).

Analyzing one or more cells to determine the existence of a condition or disease can also include detecting mitochondrial DNA, telomerase, or a nuclear matrix protein in the enriched rare cell sample; detecting the presence or absence of perinuclear compartments in a cell of the enriched sample; or performing gene expression analysis, determining nucleic acid copy number, in-cell PCR, or fluorescence in-situ hybridization of the enriched sample.

In any of the embodiments herein, target nucleic acids can be obtained from a single cell.

The fetal cell-enriched sample can be “binned” prior to analysis of the one or more enriched cells. Binning is any process which results in the reduction of complexity and/or total cell number of the enriched cell output. Binning can be performed by any method known in the art or described herein. One method of binning the enriched cells is by serial dilution. Such dilution can be carried out using any appropriate platform (e.g., PCR wells, microtiter plates). Other methods include nanofluidic systems which separate samples into droplets (e.g., BioTrove, Raindance, Fluidigm). Such binning can result in the presence of a single cell present in a well or nanodroplet. When binning a sample enriched for fetal cells, preferably each site includes 0 or 1 cell.

Binning can be preceded by fetal cell enriching methods including, but not limited to affinity binding (e.g., using lectin, anti-MMP14 and/or anti-CD71 antibodies). For example, an enriched maternal blood can be subjected to gradient centrifugation, lectin-based affinity separation and MMP-14-based affinity separation. An aliquot of this sample containing approximately 100 cells can then be separated into 100 bins (PCR wells or other acceptable binning platform), such that each bin would be expected to contain approximately one cell. One of skill in the art will recognize that the number of bins can be increased depending on experimental design and/or the platform used for binning. The reduced complexity of the binned cell populations can facilitate further genetic and cellular analysis of the target cells.

In some embodiments, analysis is performed on individual bins to confirm the presence or absence of a nucleated fetal cell in the individual bin. Such analysis can be performed using any method known in the art in accordance with the teachings herein, including, but not limited to, FISH, PCR, STR detection, SNP analysis, biomarker detection, and sequence analysis.

Fetal conditions that can be determined based on the methods and systems herein include the presence of a fetus and/or a condition of the fetus such as fetal aneuploidy e.g., trisomy 13, trisomy 18, trisomy 21 (Down Syndrome), Klinefelter Syndrome (XXY) and other irregular number of sex or autosomal chromosomes, including monosomy of one or more chromosomes (X chromosome monosomy, also known as Turner's syndrome), trisomy of one or more chromosomes (13, 18, 21, and X), tetrasomy and pentasomy of one or more chromosomes (which in humans is most commonly observed in the sex chromosomes, e.g., XXXX, XXYY, XXXY, XYYY, XXXXX, XXXYY, XYYYY and XXYYY), monoploidy, triploidy (three of every chromosome, e.g., 69 chromosomes in humans), tetraploidy (four of every chromosome, e.g., 92 chromosomes in humans), pentaploidy and multiploidy. Other fetal conditions that can be detected using the methods herein include segmental aneuploidy, such as 1p36 duplication, dup(17)(p11.2p11.2) syndrome, Down syndrome, Pre-eclampsia, Pre-term labor, Edometriosis, Pelizaeus-Merzbacher disease, dup(22)(q11.2q11.2) syndrome, Cat eye syndrome. In one embodiment, the fetal abnormality to be detected is due to one or more deletions in sex or autosomal chromosomes, including Cri-du-chat syndrome, Wolf-Hirschhorn syndrome, Williams-Beuren syndrome, Charcot-Marie-Tooth disease, Hereditary neuropathy with liability to pressure palsies, Smith-Magenis syndrome, Neurofibromatosis, Alagille syndrome, Velocardiofacial syndrome, DiGeorge syndrome, steroid sulfatase deficiency, Kallmann syndrome, Microphthalmia with linear skin defects, Adrenal hypoplasia, Glycerol kinase deficiency, Pelizaeus-Merzbacher disease, testis-determining factor on Y, Azospermia (factor a), Azospermia (factor b), Azospermia (factor c) and 1p36 deletion. In one embodiment, the fetal abnormality is an abnormal decrease in chromosomal number, such as XO syndrome. The above list serves merely as an example of possible genetic diseases that can be assessed using the methods provided herein. However, these methods can extend to any disease with a known genetic cause including, but not limited to, diseases in public databases such as OMIM.

Identification of Fetal Cell Sequence or Expression

In some embodiments, the methods provided herein further comprise analyzing the nucleotide sequence of a nucleic acid molecule data or gene expression data determined for individual cells and determining whether or not the data is indicative of a maternal sequence or gene expression or a fetal sequence or gene expression. In some such embodiments, the probability of the data being indicative of maternal or fetal information is determined. It is contemplated that, even after enrichment methods are performed, maternal cells are likely to still be present in the fetal cell-enriched sample. Thus, methods can be implemented for identifying a cell as being of fetal or maternal origin, or for identifying the probability of a cell as being of fetal or maternal origin. The methods provided herein permit an enhanced ability to identify a cell as being of fetal origin or identify the likelihood of a cell as being of fetal origin because the analysis of the cells of the fetal cell-enriched sample is performed on a single cell-by-single cell basis. Accordingly, the determination of a sequence or gene expression in accordance with embodiments provided herein is not based on an ensemble of cells or average signal from a number of cells, but instead is based on a plurality of single cell sequence or gene expression measurements.

In some embodiments, the methods include analyzing the nucleotide sequence of a nucleic acid molecule data or gene expression data determined for individual cells for each of two or more cells of the fetal cell-enriched sample comprises. Such methods can include classifying each cell as belonging to a first population of cells or a second population of cells based on the determined nucleotide sequence or gene expression, and identifying probabilities of the first and second populations of cells being of fetal or maternal origin. In some such embodiments, the probabilities of the first and second populations of cells being of fetal or maternal origin are identified by comparing the nucleotide sequence of a nucleic acid molecule or expression of a gene for each of the first and second populations to a known nucleotide sequence of a nucleic acid molecule or expression of a gene for known maternal cells, wherein the population of cells bearing a higher nucleotide sequence or gene expression similarity to the known maternal cells is identified as being of maternal origin.

In some embodiments, the probabilities of the first and second populations of cells being of fetal or maternal origin are identified by assessing the size of the first and second populations of cells, wherein the larger population of cells is identified as being of fetal origin. It is contemplated herein that some embodiments of the methods provided herein yield a fetal cell-enriched sample that contains more fetal cells than maternal cells. Thus, in these embodiments, while maternal cells are likely to nevertheless be present in a fetal cell-enriched sample, the sequence or gene expression more common over the entire population of cells of the fetal cell-enriched sample is more likely to be that of fetal cells and not maternal cells. Accordingly, the methods provided herein, while the can be applied to a measurement of a single cell, have an increased probability of accuracy when applied to a measurement of a plurality of cells. For example at least 2, 3, 4, 5, 6, 7, 8, 9, 10, 11, 12, 13, 14, 15, 16, 17, 18, 19 or 20 individual cells can be analyzed for nucleotide sequence information or for gene expression information, and the results of this analysis can provide an identification of a fetal nucleotide sequence or gene expression and/or a probability of a particular nucleotide sequence or gene expression being of fetal origin.

In some embodiments, a diagnosis is made by comparing results from such genetic analyses with results from similar analyses from a reference sample (e.g., maternal cells). For example, a fetal cell enriched sample can be analyzed to determine the presence of one or more fetal cells and/or a sequence or gene expression in such cells by comparing the sequence or gene expression in cells from a fetal cell enriched sample to sequence or gene expression in known maternal cells (e.g., maternal dermal or epithelial cells).

In one embodiment transcript or protein expression of a gene in a fetal cell can be used as a marker to enrich, enumerate, purify, detect or identify the fetal cell if the expression of the gene is higher or lower in the fetal cell than in a reference sample, e.g., in a maternal cell. In one embodiment, a gene can be a fetal cell marker if the level of its expression (in the form of a transcript or protein) is at least about 10%, 11%, 12%, 13%, 14%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500%, 750%, 1000%, 2000%, 3000%, 4000%, 5000% or 10,000% higher or lower than the level of expression of the gene (in the form of a transcript or protein) in a reference sample (e.g., a maternal cell). In one embodiment, a gene has a higher level of protein or transcript expression in comparison to a reference sample (e.g., a maternal cell). In another embodiment, a gene can be a marker of a fetal cell if the ratio of the expression of a of protein or transcript of the gene in a fetal cell compared to the expression of the gene in a reference sample (e.g., a maternal cell) is at least about 11:10, 6:5, 13:10, 7:5, 3:2, 8:5, 17:10, 9:5, 2:1, 3:1, 4:1, 5:1, or 10:1, 20:1, 25:1, 30:1, 35:1, 40:1, 45:1, 50:1, 55:1, 60:1, 65:1, 70:1, 75:1, 80:1, 85:1, 90:1, 100:1, 150:1, 200:1, 250:1, 300:1, 350:1, 400:1, 450:1, 500:1, 550:1, 600:1, 650:1, 700:1, 750:1, 800:1, 850:1, 900:1, 950:1, or 1000:1. In another embodiment a gene can be a marker of a fetal cell if the expression of a of protein or transcript of the gene in a fetal cell is at least about 1.1-, 1.2-, 1.3-, 1.4-, 1.5-, 1.6-, 1.7-, 1.8-, 1.9-, 2-, 3-, 4-, 5-, 10-, 15-, 20-, 25-, 30-, 35-, 40-, 45-, 50-, 55-, 60-, 65-, 70-, 75-, 80-, 85-, 90-, 95, or 100-fold higher or lower than expression of the transcript in a reference sample (e.g., a maternal cell). Levels of transcript or protein expression can be normalized to expression levels of other transcripts, or proteins, respectively.

### Examples

Samples were obtained from 100 pregnant women in their 1^{st}, 2^{nd} and/or 3^{rd} trimester of pregnancy. Blood samples were processed within 3 hours after collection. Each blood sample was diluted 1:1 with phosphate-buffered saline (PBS) to appropriate volume. The diluted blood sample was layered over density media adjusted to a density of ˜1.09 g/ml. Density centrifugation was performed at 1500 rpm for 30 min at room temperature. After washing 3 times with cold PBS, mononuclear cells were collected by centrifugation again for 10 min at room temperature. The sediment was resuspended with PBS and subjected to lectin selection using soybean agglutinin (SBA) as described in Kitagawa et al., 2002 Prenat. Diagn 22:17-21.

10 mg of protein A/G magnetic beads mixture (Life Technologies Corporation) were resuspended and washed twice with PBS plus Tween-20. 50 μg of candidate antibodies (CD71 and MMP14; EMD Millipore Corporation) were added to 400 μl beads mixture diluted in PBS plus Tween-20, incubated and rotated at room temperature for 30 min. Beads coupled with antibody were separated with magnetic stand and washed for 3 times in PBS plus Tween-20. Crude mononuclear cells isolated after lectin selection were added to antibody coupled protein A/G beads diluted in 400 μl PBS plus Tween-20 and incubated for 1 hour at room temperature. After washing, enriched cells were diluted into 20 μl for downstream analysis.

In order to estimate the quality and purity of cells after enrichment, half of the enriched cell sample is subjected to cytogenetic analysis and immune-stained with fetal cell surface markers as indicated before. For the other half of the sample, cells are separated into individual wells of a 96-well plate to ensure no more than 1 cell per well.

Cytogenetic analysis, transcriptome/genome and other analysis of DNA/RNA composition of 3-5 single cells will be performed. As a control, maternal cells will be harvest from saliva samples. The DNA sequence of single cell data will be compared with that from maternal cells to distinguish fetal single cell from maternal single cells as well as fetal cell-specific genetic variations. Differential RNA and protein expression will be computed between individual single cells, which will be utilized to identify novel marks.

1. A method of obtaining a fetal cell-enriched sample from a maternal sample comprising:
providing a maternal sample;
contacting the maternal sample with a first stationary phase having affinity for one or more saccharides;
separating components of the maternal sample bound to the first stationary phase from components of the maternal sample not bound to the first stationary phase;
retaining the components of the maternal sample bound to the first stationary phase;
contacting the maternal sample with an isolatably labeled affinity molecule having affinity for matrix metalloproteinase 14;
separating components of the maternal sample bound to the isolatably labeled affinity molecule from components of the maternal sample not bound to the isolatably labeled affinity molecule; and
retaining the components of the maternal sample bound to the isolatably labeled affinity molecule,
thereby providing a fetal cell-enriched sample.

2. (canceled)

3. (canceled)

4. (canceled)

5. The method of claim 1, wherein the first stationary phase is a lectin-bound stationary phase.

6. (canceled)

7. (canceled)

8. The method of claim 1, wherein, subsequent to retaining the components of the maternal sample bound to the first stationary phase, the retained components of the maternal sample bound to the first stationary phase are contacted with the isolatably labeled affinity molecule having affinity for matrix metalloproteinase 14.

9. The method of claim 1, further comprising:
contacting the maternal sample with a second stationary phase having affinity for a fetal cell surface marker other than matrix metalloproteinase 14;
separating components of the maternal sample bound to the second stationary phase from components of the maternal sample not bound to the second stationary phase; and
retaining the components of the maternal sample bound to the second stationary phase.

10. The method of claim 9, wherein the fetal cell surface marker is selected from the group consisting of transferin receptor (CD71), glycophorin A (GPA), EGFR, thrombospondin receptor (CD36), CD 34, HbF, HAE 9, FB3-2, H3-3, erythropoietin receptor, HBE, AFP, APOC3, SERPINC1, AMBP, CPB2, ITIH1, APOH, HPX, beta-hCG, AHSG, APOB, J42-4-d, 2,3-biophosphoglycerate (BPG), Carbonic anhydrase (CA), and Thymidine kinase (TK).

11. (canceled)

12. (canceled)

13. (canceled)

14. The method of claim 1, further comprising:
providing the fetal cell-enriched sample; and
analyzing a nucleotide sequence of a nucleic acid molecule or expression of a gene in one or more cells from the fetal cell-enriched sample.

15. The method of claim 14, wherein the analyzing a nucleotide sequence of a nucleic acid molecule comprises sequencing genomic DNA of one or more cells from the fetal cell-enriched sample.

16. (canceled)

17. (canceled)

18. The method of claim 14, wherein the expression of a gene comprises hybridizing a detectable antibody to the surface of one or more cells from the fetal cell-enriched sample.

19. (canceled)

20. (canceled)

21. (canceled)

22. A method of assessing a maternal sample for a fetal nucleotide sequence or a fetal gene expression comprising:
providing a maternal sample;
contacting the maternal sample with a first stationary phase having affinity for one or more saccharides;
separating components of the maternal sample bound to the first stationary phase from components of the maternal sample not bound to the first stationary phase;
retaining the components of the maternal sample bound to the first stationary phase;
contacting the maternal sample with an isolatably labeled affinity molecule having affinity for a fetal cell surface marker;
separating components of the maternal sample bound to the isolatably labeled affinity molecule from components of the maternal sample not bound to the isolatably labeled affinity molecule;
retaining the components of the maternal sample bound to the isolatably labeled affinity molecule to provide an fetal cell-enriched sample for analysis,
determining a nucleotide sequence of a nucleic acid molecule or expression of a gene in individual cells for each of two or more cells of the fetal cell-enriched sample; and
assessing the nucleotide sequence of a nucleic acid molecule or expression of a gene determined for individual cells for each of two or more cells of the fetal cell-enriched sample to identify a fetal nucleotide sequence or gene expression.

23. The method of claim 21, wherein assessing the nucleotide sequence of a nucleic acid molecule or expression of a gene determined for individual cells for each of two or more cells of the fetal cell-enriched sample comprises:
classifying each cell as belonging to a first population of cells or a second population of cells based on the determined nucleotide sequence or gene expression; and
identifying probabilities of the first and second populations of cells being of fetal or maternal origin.

24. The method of claim 23, wherein the probabilities of the first and second populations of cells being of fetal or maternal origin are identified by comparing the nucleotide sequence of a nucleic acid molecule or expression of a gene for each of the first and second populations to a known nucleotide sequence of a nucleic acid molecule or expression of a gene for known maternal cells, wherein the population of cells bearing a higher nucleotide sequence or gene expression similarity to the known maternal cells is identified as being of maternal origin.

25. The method of claim 23, wherein the probabilities of the first and second populations of cells being of fetal or maternal origin are identified by assessing the size of the first and second populations of cells, wherein the larger population of cells is identified as being of fetal origin.

26. A method of assessing a maternal sample for a fetal nucleotide sequence or a fetal gene expression comprising:
providing a nucleotide sequence of a nucleic acid molecule or expression of a gene in individual cells for each of two or more cells of the fetal cell-enriched sample, wherein the fetal cell-enriched sample has been prepared by a method comprising:
providing a maternal sample;
contacting the maternal sample with a first stationary phase having affinity for one or more saccharides;
separating components of the maternal sample bound to the first stationary phase from components of the maternal sample not bound to the first stationary phase;
retaining the components of the maternal sample bound to the first stationary phase;
contacting the maternal sample with an isolatably labeled affinity molecule having affinity for a fetal cell surface marker;
separating components of the maternal sample bound to the isolatably labeled affinity molecule from components of the maternal sample not bound to the isolatably labeled affinity molecule; and
retaining the components of the maternal sample bound to the isolatably labeled affinity molecule to provide an fetal cell-enriched sample for analysis; and
assessing the nucleotide sequence of a nucleic acid molecule or expression of a gene determined for individual cells for each of two or more cells of the fetal cell-enriched sample to identify a fetal nucleotide sequence or gene expression.

27. The method of claim 26, wherein the probabilities of the first and second populations of cells being of fetal or maternal origin are identified by comparing the nucleotide sequence of a nucleic acid molecule or expression of a gene for each of the first and second populations to a known nucleotide sequence of a nucleic acid molecule or expression of a gene for known maternal cells, wherein the population of cells bearing a higher nucleotide sequence or gene expression similarity to the known maternal cells is identified as being of maternal origin.

28. The method of claim 26, wherein the probabilities of the first and second populations of cells being of fetal or maternal origin are identified by assessing the size of the first and second populations of cells, wherein the larger population of cells is identified as being of fetal origin.

29. The method of claim 22, wherein the fetal cell-enriched sample preparation method further comprises, prior to contacting the maternal sample with said first stationary phase and said first stationary phase:
separating components of the maternal sample according to size and/or density; and
harvesting the separated components of the maternal sample having the size and/or density of nucleated fetal red blood cells.

30. (canceled)

31. (canceled)

32. The method of claim 22, wherein the first stationary phase is a lectin-bound stationary phase.

33. (canceled)

34. (canceled)

35. The method of claim 22, wherein, subsequent to retaining the components of the maternal sample bound to the first stationary phase, the retained components of the maternal sample bound to the first stationary phase are contacted with the isolatably labeled affinity molecule.

36. The method of claim 22, wherein the fetal cell-enriched sample preparation method further comprises:
contacting the maternal sample with a second stationary phase having affinity for a fetal cell surface marker;
separating components of the maternal sample bound to the second stationary phase from components of the maternal sample not bound to the second stationary phase; and
retaining the components of the maternal sample bound to the second stationary phase.

37. The method of claim 36, wherein the fetal cell surface marker is selected from the group consisting of MMP14 (matrix metalloproteinase 14), transferin receptor (CD71), glycophorin A (GPA), EGFR, thrombospondin receptor (CD36), CD 34, HbF, HAE 9, FB3-2, H3-3, erythropoietin receptor, HBE, AFP, APOC3, SERPINC1, AMBP, CPB2, ITIH1, APOH, HPX, beta-hCG, AHSG, APOB, J42-4-d, 2,3-biophosphoglycerate (BPG), Carbonic anhydrase (CA), and Thymidine kinase (TK).

38. (canceled)

39. (canceled)

40. (canceled)

41. The method of claim 22, wherein the fetal cell-enriched sample preparation method further comprises:
providing the fetal cell-enriched sample; and
analyzing a nucleotide sequence of a nucleic acid molecule or expression of a gene in two or more cells from the fetal cell-enriched sample.

42. The method of claim 41, wherein the analyzing a nucleotide sequence of a nucleic acid molecule comprises sequencing genomic DNA of two or more cells from the fetal cell-enriched sample.

43. (canceled)

44. (canceled)

45. The method of claim 41, wherein the expression of a gene comprises hybridizing a detectable antibody to the surface of two or more cells from the fetal cell-enriched sample.

46. (canceled)

47. (canceled)

48. (canceled)
